(19) United States
(12) Patent Application Publication
(10) Pub. No.: US 2025/0205484 A1
(43) Pub. Date: Jun. 26, 2025

(54) Apparatus and Method for Determining Phase of Gait of a Subject

(71) Applicant: BIOMEX LTD., London (GB)

(72) Inventors: Lance Rane, London (GB); Anthony Michael James Bull, London (GB); Eline Van Der Kruk, Rijswijk (NL)

(73) Assignee: BIOMEX LTD., London (GB)

(21) Appl. No.: 18/848,433

(22) PCT Filed: Mar. 14, 2023
(86) PCT No.: PCT/IB2023/052474

§ 371 Date: Sep. 18, 2024

(30) Foreign Application Priority Data
Mar. 18, 2022 (GB) 2203855.8

(51) Int. Cl. A61N 1/36

(52) U.S. Cl. CPC A61N 1/36003; A61N 1/36031

(57) ABSTRACT

An aspect of the invention providing a method for determining the phase of gait of a subject is provided, the method comprising: transmitting from at least one motion sensor, data corresponding to motion of a part of a lower limb of the subject to a control unit; estimating a gait phase of the subject using the control unit; and sending an input signal based on the estimated gait phase to a controller; based on the input signal, executing a pre-determined instruction or action.

### FIELD

Aspects of the present invention relate to apparatus and methods for determining phase of gait of a subject.

### BACKGROUND

The human gait cycle is typically broken down into the stance phase, i.e., the period of time during which a foot is on the ground, and the swing phase, i.e., the period during which the foot is in the air. As a person walks, one foot will always be in the stance phase and the other will be in the swing phase. When running the gait cycle includes a period during which both feet are in the swing phase.

It is beneficial to understand the phase of the human gait cycle for many different applications. This is typically achieved using pressure sensors to determine the occurrence of heel strike, toe off and flat foot. These events can then be used in conjunction with a measurement of cadence to determine a sub-set of gait phases that fall within the stance and swing phases. In an evaluation setting, a combination of video and sensor data may be used to enable a medical professional to determine deficiencies in a patient's gait cycle.

Many robotic devices are based on the human anatomy. It is therefore useful for such devices to have an intelligent understanding, or programming, of the gait cycle of each limb of the robotic device. Such data is used as part of the control strategy for each limb of a robot. For example, a robot comprising eight legs must be operated in such a way that each leg is controllable independently but in relation to each other leg. This is only possible through an understanding of the gait cycle of the robotic device.

Aspects of the present invention seek to provide improved systems and methods for determining phase of gait.

### SUMMARY

As used herein, the term inertial measurement unit (or IMU) shall be interpreted as meaning an electronic device that is used to measure and report data representative of a specific force, angular rate, and orientation of an object, i.e., the limb of a subject. IMUs as used in embodiments of the invention comprise at least one accelerometer and at least one gyroscope. In some embodiments, IMUs incorporated in the present invention may also include a magnetometer.

As used herein, the term functional electrical stimulation (or FES) shall be interpreted as meaning the application of an electrical charge to the muscles of a subject for the purposes of rehabilitation and injury prevention. As discussed throughout this description, application of FES is just one example of how the present invention may be implemented.

Aspects of the present invention relate generally to the determination of phase of gait of a subject using novel apparatus and computational techniques. This is demonstrated by way of reference to a specific application of applying FES based on phase of gait. During the gait cycle, different muscles are activated depending on the current phase of gait. Accurately determining or predicting the phase of gait is therefore imperative to ensure effective muscle targeting using FES. Aspects of the present invention achieve this through use of a neural network stored on a device that has been trained to accurately predict the subject's phase of gait from IMU data. Data collected by the IMU may be transmitted to a control unit. The control unit may estimate a gait phase using novel computational techniques. The estimated gait phase may be used to determine when to apply FES to a patient's muscles. For example, if the gait phase cycle is split into 13 phase segments, the control unit may transmit an input signal to a FES controller consistent with an instruction to apply FES at phase segments 2-4 and 9-11, or as specified by a physician. The FES controller may apply FES to the patient's muscles in accordance with instructions received from the control unit. In other examples, the determined phase of gait may be utilised to execute other actions. In an example of a robot, the determined phase of gait may be utilised as part of a multi-limb motion control strategy. Details of such an embodiment are outside of the scope of this invention but it will be appreciated that certain aspects and embodiments of the present invention may be suitable for use in such application.

It will also be appreciated, that while the present description focuses on motion of a lower limb subject, aspects, and embodiments of the present invention applicable to determination of a position or phase of motion of an upper limb of the subject. In some aspects and embodiments of the invention, the position of phase of only a part of an upper or lower limb subject may be determined. For example, certain applications may only be interested in the phase or position of an upper part of a leg of a subject. However, data received regarding motion of the upper part of a leg of a subject may also be extrapolated and interpreted to predict the phase or position of a lower part of the leg of the subject. That is the focus of the embodiments described in this description.

In one aspect of the invention a method for determining the phase of gait of a subject is provided, the method comprising: transmitting from at least one motion sensor, data corresponding to motion of a part of a lower limb of the subject to a control unit; estimating a gait phase of the subject using the control unit; and sending an input signal based on the estimated gait phase to a controller; based on the input signal, executing a pre-determined instruction or action.

The gait phase of the subject may generally be divided into phases identified as a stance phase and a swing phase. It is advantageous to be able to not only determine between the stance phase and a swing phase, but also between sub phases thereof. It is known that at each stage of the subject's gait cycle different muscles may be activated. Accurate determination of each sub-phase of a subject's gait cycle enables the application of certain actions to the subject. For example, it may be advantageous to implement a particular action at a midpoint of the swing phase of the subject's gait cycle. Such a possibility is provided for by the present invention.

In one embodiment, the pre-determined instruction or action comprises applying functional electrical stimulation (FES) to the lower limb of the subject.

FES is commonly used in connection with rehabilitation from injury. Application of FES to a particular muscle group can be used to restore or improve muscle function. It has been postulated that activation of a muscle group in combination with application of FES may not only be used for rehabilitation from injury, but it may also have performance benefits and the advantage of reducing the occurrence of injury in the first place. The present invention enables the identification of a point within a subject's gait cycle where target muscle groups are activated. This knowledge enables FES To be applied to a subject's limb at a defined point in the gait cycle.

In one embodiment, the step of estimating the gait phase of the subject using the control unit comprises: assigning a phase vector to the data received by the control unit from at least one motion sensor; determining the probability of the phase vector lying within each gait phase of the subject; estimating the gait phase of the subject by selecting the gait phase having the highest probability of the phase vector lying therein.

In one embodiment, the phase vector is assigned based on a combination of data captured by the one or more motion sensors that is representative of linear and rotational motion in x, y and z axes.

In one embodiment, the data received by the control unit from one or more motion sensors is transformed through a neural network stored in non-volatile memory in communication with the control unit to transform the raw data from the one or more motion sensors into the phase vector.

In one embodiment, the neural network comprises a plurality of baseline gait phase vectors against which the phase vector is compared and assigned a similarity score, and wherein the similarity score is used to define a probability distribution over a plurality of gait phase labels to determine the likelihood of the phase vector lying within a gait phase represented by each of the gait phase labels.

In one embodiment, the method further comprises the step of selecting the gait phase label having the maximum probability of the phase vector lying therein.

In one embodiment, the selected gait phase identifies whether the gait phase of the subject is stationary, in a stance phase, or in a swing phase. In one embodiment, at least the stance phase or swing phase is sub-divided into a plurality of sub-gait phases represented by the gait phase labels. In another embodiment, both stance phase and swing phase are sub-divided into a plurality of sub-gait phases represented by the gait phase labels.

In one embodiment, the stance phase of the subject's gait cycle is sub-divided into sub-phases 1-6. In one embodiment, the swing phase of the subject's gait cycle is sub-divided into sub-phases 7-12. In another embodiment, the plurality of gait phase labels further comprises a gait phase label 13 that is representative of the subject either having both feet on the floor or undertaking an activity that does not involve walking or running.

In one embodiment, the step of applying FES to a lower limb of the subject comprises the FES controller applying FES at a first one or more gait phases of the subject. In one embodiment, FES is applied to one or more gait phases of the subject during each gait cycle either individually or in groups. In one embodiment, an intensity, or another parameter, of FES is variable depending on the estimated gait phase of the subject.

In another aspect of the invention, a method of estimating gait phase of a subject is provided, the method comprising: transmitting from at least one motion sensor to a control unit, data corresponding to motion of a part of a lower limb of the subject; assigning a phase vector to the data received by the control unit from the at least one motion sensor; determining the probability of the phase vector lying within each gait phase of the subject's gait cycle; estimating the gait phase of the subject by selecting the gait phase having the highest probability of the phase vector lying therein.

In another aspect of the invention, apparatus for estimating phase of gait of a subject comprising: at least one motion sensor associated with the limb of a subject; a control unit; non-volatile memory; and communication means, wherein, the at least one motion sensor is configured to receive data associated with motion of the limb of the subject, said data being transmitted to the control unit and stored in the non-volatile memory, and wherein external data representative of an action to be undertaken at a predetermined phase of gait is receivable by the communication means and stored in the non-volatile memory, wherein the controller is configured to determine the phase of gait from the received data and undertake the action in accordance with the predetermined phase of gait.

In one embodiment, the apparatus further comprises a support substrate to which each of the at least one motion sensor, controller, non-volatile memory, and communication means are integrated, the support substrate being configured for selective insertion and removal to/from an item of clothing.

In one embodiment, the apparatus further comprises means for applying functional electrical stimulation (FES) to the subject's limb. In one embodiment, the means for applying FES is variable in terms of voltage and duration.

In one embodiment, the at least one motion sensor comprises at least one IMU. In one embodiment, the at least one IMU is supported by a rigid backing plate.

Further areas of applicability of the present invention will become apparent from the detailed description provided hereinafter. The detailed description and specific examples, while indicating the preferred embodiment of the invention, are intended to be given by way of example only.

### FIGURES

Aspects and embodiments of the invention will now be described by way of reference to the following figures.

FIG. 1 illustrates an article of clothing embodying aspects and embodiments of the invention.

FIG. 2 illustrates data captured by IMUs according to aspects and embodiments of the invention.

FIG. 3 illustrates the raw data collected by an IMU of aspects and embodiments of the invention.

FIG. 4 illustrates a phase vector within label space according to aspects and embodiments of the invention.

FIG. 5 illustrates an example system architecture according to aspects and embodiments of the invention.

FIG. 6 is a flow chart detailing a method of applying FES to a subject according to an aspect of the invention.

FIG. 7 is a flow chart detailing a method of estimating the gait phase of a subject according to another aspect of the invention.

### DESCRIPTION

The following description of the preferred embodiment(s) is merely exemplary in nature and is in no way intended to limit the invention, its application, or uses.

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivatives thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

Reference herein to FES shall not be interpreted as limiting to the potential applications of aspects of the invention. For example, in certain aspects and embodiments of the invention the determined phase of gait of the subject may be utilised for other purposes. As an example, the determined phase of gait may be used to identify the position of a robotic limb. This may be helpful in identifying a particular point in the gait cycle where another specified action may be required. Furthermore, the determination of a phase of gait of a subject may be helpful in collection of data for physical performance metrics, providing a prognosis or diagnosis of injury or condition in connection with extraneous data. It will be appreciated that aspects and embodiments of the invention may be utilised in multiple different applications. The reference to FES herein is just one application of the present invention.

Discussion herein of gait phase labels and gait phase vectors, shall be interpreted as follows. A gait phase label for the purposes of this description shall be interpreted as a data label between 1-n that represents a phase of gait of a subject. By reference to subject, the description is not limited to a person or a limb of a person. Such reference could equally apply to the limb of an animal or a robotic limb. A gait phase vector for the purposes of this description shall be interpreted as a representation in multidimensional space of motion data collected by at least one IMU that is representative of the position of phase of a subject's limb as part of its gait cycle. The gait phase vector may be used in embodiments of the present invention to determine a particular phase of gait of the subject which can be identified by a specific gait phase label. This will become apparent by reference to the remainder of this description.

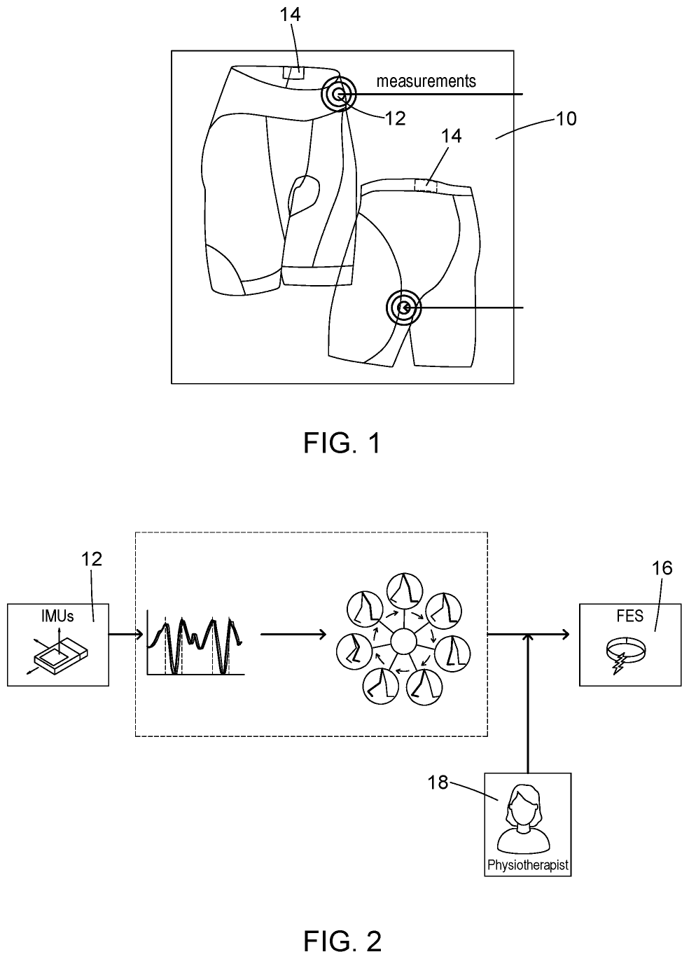

As shown in FIG. 1, an embodiment of the invention comprises an article of clothing (10), i.e., pair of shorts, which comprise an IMU (12) which is connected to a control unit (14) by way of wiring integrated into the article of clothing (10) or by wiring connecting various components of the invention via a removable exoskeleton (as discussed further below). Some embodiments of the invention may comprise a shirt, leggings, vest, socks, or any other item of clothing.

The IMU (12), as illustrated in FIG. 2, comprises at least one of each of a gyroscope and accelerometer to measure acceleration and rotation in the x, y and z dimensions, providing six degrees of freedom per sample. In one embodiment, the sampling interval is 1 ms, such that 1000 such samples are collected per second. The IMU (12) may be expanded to 9-DOF through inclusion of data received from a magnetometer and the collection of additional magnetic field strength data. Multiple samples representing a time window of some prescribed length encompassing the totality of data measured by the IMU through repeated sampling are stored in volatile memory. As new data are collected with the passing of subsequent sampling intervals, they are appended to this storage, with displacement of the oldest of the existing samples, such that the size of the storage is kept constant. These stored data are used at intervals to assign a phase vector to the data collected by the IMU (12).

The IMU (12) is supported by a polyurethane backing so that the relative orientation of the IMU (12) with the subject is generally consistent. In some embodiments, one IMU (12) may be provided in association with a single leg of the subject. In other embodiments, two IMUs (12) may be provided in association with respective legs of the subject. In other embodiments, more than one IMU (12) may be associated with each leg of the subject.

The control unit (14) is housed within a control box which further comprises non-volatile memory and communication means. The control box may be referred to as a brain and may comprise control circuitry that incorporates the control unit (14), the non-volatile memory and communication means. The non-volatile memory receives and stores data from the IMU and communication means. The communication means comprises one or more of a WiFi, ANT+ or Bluetooth module, for example, to enable communication with an external computing device. Optionally, the control unit (14) may connect to external sensors, i.e., pressure or positioning sensors, by way of the communication means and/or via a cabled connection. Such external sensors may be used to further refine and/or improve the accuracy of the prediction of phase of gait of a subject. For example, if pressure sensors provided within a shoe and insole are incorporated into the system, the control unit (14) will have specific reference points to determine when each foot is in contact with a ground surface or not in contact with a ground surface. Such additional data may provide relative certainty as to whether each limb of the subject is either in a stance phase or a swing phase. As described herein, each of the stance phase or the swing phase may be divided into n sub phases that are identified by respective gait phase labels.

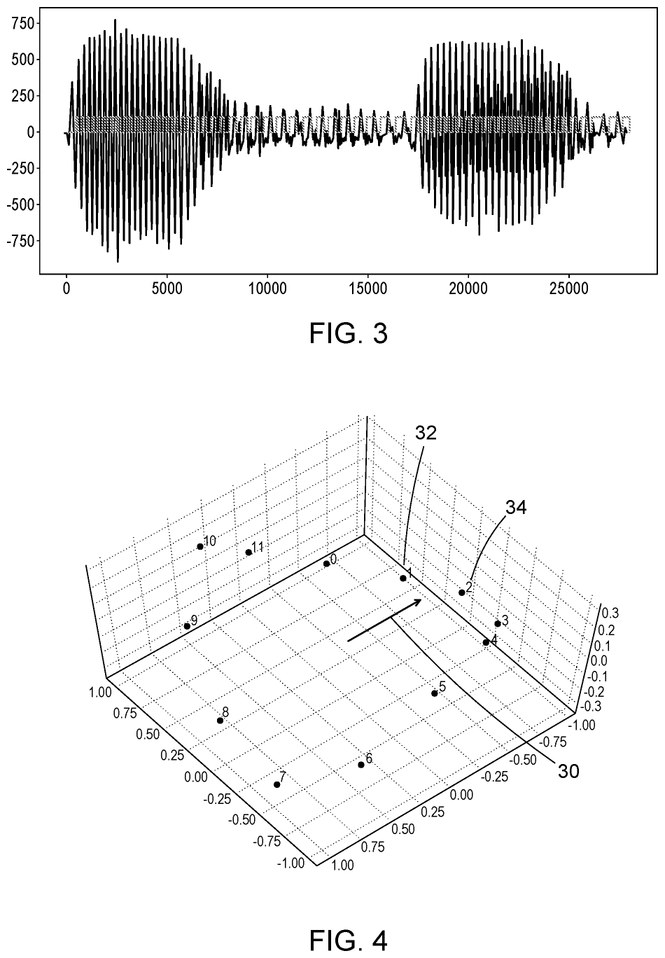

The non-volatile memory may receive and store a trained neural network from an external computer device. The neural network may be trained using motion data recorded from a plurality of subjects who each wear an IMU (12) on each of their thighs and/or a contact sensor on each forefoot and heel. Each IMU (12) records linear acceleration in the x, y and z dimensions and angular velocity, around the same axes. Each contact sensor records whether the forefoot or heel, as applicable, of each foot is in contact with a ground surface or not in contact with a ground surface. The data collected using the IMUs (12) and contact sensors is used to train a neural network to predict the phase of the subject's gait cycle. In one embodiment, the gait cycle is represented in a finite number of phases from 1-n. In one embodiment, n=13. As illustrated in FIG. 3, the raw data is collected by the IMUs (12) and contact sensors. In this example, the angular velocity in the z axis and the time which the subject's foot is in contact with a ground surface is displayed. The greater the angular velocity, the shorter the time that the subject's foot is in contact with the ground surface and vice versa. Furthermore, the data received from the contact sensors may be segregated, to determine an approximate gait phase within a range of gait phases during the time in which the subject's foot is in either the stance phase or the swing phase. For example, the gait cycle may first be segregated into the stance phase, i.e., phases 1 to 6, and the swing phase, i.e., phases 7 to 12, by subdividing each of stance and swing into segments of equal duration. During the first 50% (i.e., 0-50%) of the stance phase, it may be deduced that the subject is presenting a gait phase identified by labels 1-3. During the second 50% (i.e., 51-100%) of the stance phase it may be deduced that the subject is presenting a gait phase identified by labels 4-6. During the first 50% (i.e., 0-50%) of the swing phase it may be deduced that the subject is presenting a gait phase identified by labels 7-9. During the final 50% (i.e., 51-100%) of swing phase it may be deduced that the subject is presenting a gait phase identified by labels 10-12. It may also be deduced that when the subject has both feet on the ground and/or is stationary, the subject is presenting a gait phase identified by label 13.

During training, the neural network is populated with a plurality of baseline gait phase vectors that are derived from IMU data collected from multiple subjects. Each baseline gait phase vector is associated with an identified label that represents a specific phase of gait between 1 and 13. Of course, any other number of gait phase labels may also be used within the scope of the present invention. Embodiments of the invention may utilise machine learning or artificial intelligence to aggregate the data collected and generate the baseline gait phase vectors.

In operation, at regular intervals of time, data from the IMUs (12) are processed and transformed to establish a phase vector pertaining to the instantaneous motion of the subject and taking into account data measured during a recent time window. The phase vector is used to compute a probability distribution over all the labels 1-13 such that each value associated with each label indicates a probability that the phase vector lies within the gait phase represented by that label. Using this information, phase vector data may be stored within the neural network for comparison against sensor data measured by the IMUs, in use. In computing the phase vector, stored data from one or more previous time points may be used in addition to the most recently sampled data. This approach helps improve the signal to noise ratio and improve the accuracy of the predicted gait phase.

The IMUs (12) consistently record motion data to determine when a subject starts and stops walking/running. For example, the system may process recorded data sampled over a time window extending back 250 ms from the present moment, and from that recorded data determine from the derived probability distribution over gait phase labels that the subject has started walking/running. In other examples, the time window of data used to produce this distribution may be greater than or less than 250 ms. Said motion data is transmitted from the IMUs (12) to the control unit (14). The control unit (14) accesses the neural network stored in non-volatile memory and transforms said sensor data into a phase vector (30) as described herein. The phase vector (30) may represent a probability distribution over all the gait phase labels 1-13 and the label with the highest probability is selected as the predicted gait phase as an instantaneous determination.

In some embodiments, the phase vector (30) may be compared to a plurality of baseline gait phase vectors stored in the neural network to assign a similarity score for the phase vector (30) against each baseline gait phase vector. Through further processing, a probability distribution is assigned over gait phase labels 1 to 13 for the phase vector (30) and a label may be selected based on the maximum probability of the phase vector (30) lying within a gait phase identified by a particular label. As shown in FIG. 4, a phase vector (30) is illustrated as lying somewhere between gait phase labels 1-2 (32, 34). A probability of the phase vector (30) lying within a gait phase identified by each gait phase label may be determined by reference to the similarity score between the subject's phase vector (30) and each of the baseline phase vectors. In some embodiments, the gait phase label having the highest probability of the phase vector lying within the represented gait phase may be selected. In other embodiments, extraneous data such as pressure or foot sensor data may also be combined with the phase vector to further refine the probability distribution over each of the gait phase labels.

To determine that a subject is walking or running, the control unit (14) considers both instantaneous data and historical data measured by the IMUs (12). If it is determined that the entropy of the probability distribution over the gait phase labels exceeds some pre-determined threshold, it may be determined that the subject is not walking or running and instead is undertaking some other activity. In contrast, if the control unit (14) determines that the entropy is sub-threshold, it may be determined that the subject is walking or running.

In some embodiments, the control unit may retrieve historic sensor data from the non-volatile memory and compare this against presently received sensor data from the IMUs (12). If there is a noticeable discrepancy between the historic sensor data and presently received sensor data, the control unit (14) may apply a correction factor to calibrate the presently received data. In other embodiments, the control unit (14) may compare presently received sensor data against historic sensor data received from multiple subjects and stored in the neural network or remotely to determine which subject is currently being assessed and/or treated. The historic sensor data may also be used to remove noise from data received by the IMUs (12). This is described in more detail above. While not shown in the figures, the IMU(s) (12), control box, FES apparatus (16) and associated wiring may be incorporated into an exoskeleton that may be removed from the pair of shorts (10), or other article of clothing for washing purposes. A single exoskeleton may therefore be used between subjects by fitting into the shorts (10), or other article of clothing, on an as required basis. This may be advantageous in a clinical setting, for example. The FES apparatus (16) receives control signals from the control unit (14) and/or the communication means. In some embodiments, the non-volatile memory stores control signals from an external computing device. A medical professional may input into a computer application commands for electrical stimulation of the subject's muscles at a particular phase of gait. For example, the computer application may present for display a graphical representation of a gait cycle having gait phases 1-13. The medical professional may input into the computer application that FES should be applied at gait phases 2-4 and 8-10, for example. The medical professional may also input an intensity value for each gait phase. In some embodiments, FES may be permanently applied to the subject through all gait phases and the intensity varied up and down according to the medical professional's instructions.

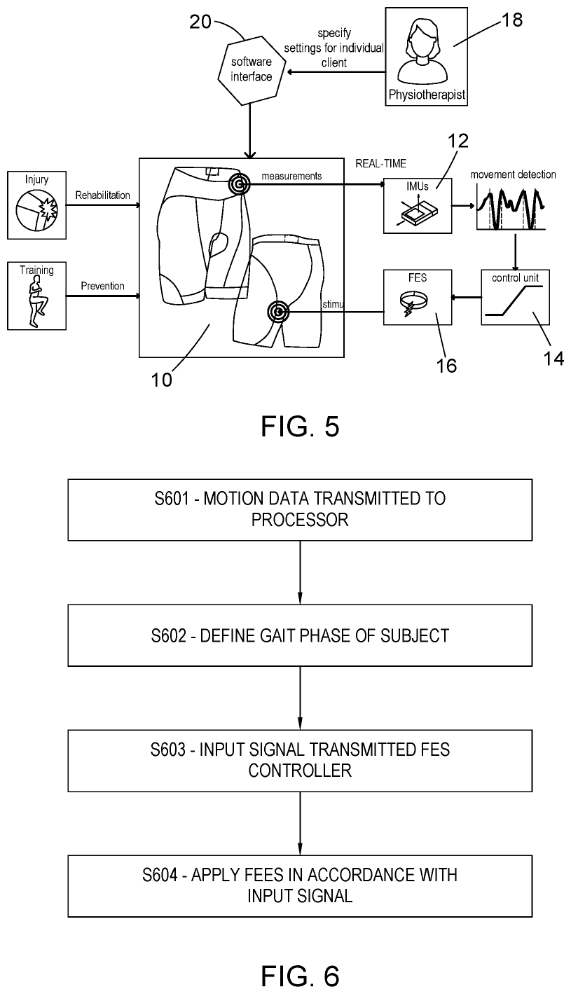

FIG. 5 shows a generalized embodiment of the present invention. Control unit (14) may receive data via an input/output (hereinafter “I/O”) path. I/O path may provide data (e.g., internet content, content over a local area network (LAN) or wide area network (WAN), and/or other content) and data to control the control unit (14), which includes processing circuitry and non-volatile memory. The control unit (14) may be used to send and receive commands, requests, and other suitable data using I/O path. I/O path may connect the control unit (14) to one or more communications paths (described below). I/O functions may be provided by one or more of these communications paths.

As referred to herein, processing circuitry should be understood to mean circuitry based on one or more microcontroller units, digital signal control units, programmable logic devices, field-programmable gate arrays (FPGAs), application-specific integrated circuits (ASICs), etc. and may include a multi-core control unit (e.g., dual-core, quad-core, hexa-core or any suitable number of cores) or supercomputer. In some embodiments processing circuitry may be distributed across multiple separate control units or processing units, for example multiple of the same type of processing units (e.g., two Intel Core i7 control units) or multiple different control units (e.g., an Intel Core i5 control unit and an Intel Core i7 control unit). In some embodiments, processing circuitry executes instructions for receiving sensor data and applying FES to a subject, wherein such instructions are stored in non-volatile memory.

In client-server-based embodiments, processing circuitry may include communication means suitable for communication with an external computing device server or other networks or servers. The instructions for carrying out the above-mentioned functionality may be stored in the non-volatile memory or on the external computing device. Processing circuitry may include a cable modem, an integrated services digital network (ISDN) modem, a digital subscriber line (DSL) modem, a telephone modem, Ethernet card, or a wireless modem for communications with other equipment, or any other suitable communications circuitry such as WiFi or Bluetooth components. Such communications may involve the Internet or any other suitable communications networks or paths. In addition, communications means may include circuitry that enables peer-to-peer communications of external computing devices, or communication of external computing devices, or communication of external computing devices in locations remote from each other.

Non-volatile memory may be embodied in an electronic storage device that is part of processing circuitry. As referred to herein, the phrase “electronic storage device” or “storage device” should be understood to mean any device for storing electronic data, computer software, or firmware, such as random-access memory, read-only memory, hard drives, flash drives, SD cards, for example.

A medical professional (18) may send instructions to processing circuitry over communications means from the external computing device. Embodiments of the invention may be implemented using any suitable architecture, i.e., a software interface (20). Processing circuitry may retrieve instructions from non-volatile memory and process the instructions to perform any of the actions discussed herein. Based on the processed instructions, processing circuitry may determine what action to perform during each phase of a subject's gait cycle.

In FIG. 6 a flow chart detailing a method (600) for applying FES to a subject is presented. The method starts at step S601 wherein at least one motion sensor attached to the lower limb of a subject transmits motion data to a control unit. At step S602, the control unit uses the received motion data to estimate a gait phase of the subject. As step S603, an input signal is sent by the control unit to a FES controller, wherein the input signal is based upon the estimated gait phase of the subject. At step S604, the input signal is used by the FES controller to determine when to apply FES to the subject.

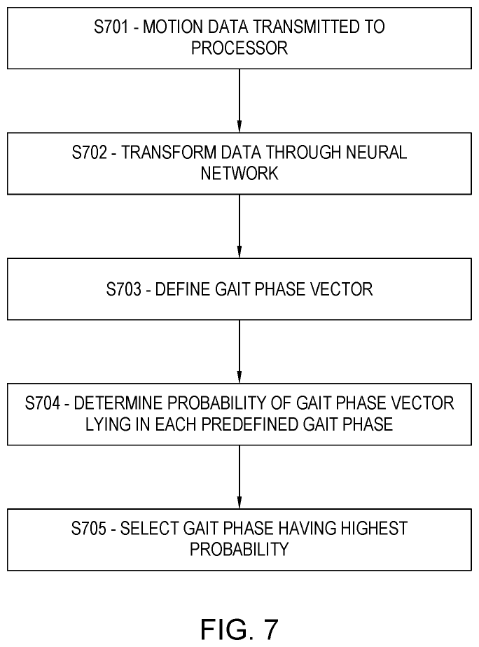

In FIG. 7 a flow chart detailing a method (700) for estimating the gait phase of a subject is presented. The method starts at step S701 wherein a motion sensor attached to the lower limb of a subject transmits motion data to a control unit. At step S702, the motion data is transformed through the neural network to define a phase vector. At step S703, the probability of the phase vector lying within each of a plurality of gait phases of the subject represented by respective gait phase labels is determined. At step S704, the gait phase of the subject is estimated by selecting the gait phase label having the highest probability of the phase vector lying therein.

It should be appreciated that in the above description of exemplary embodiments, various features are sometimes grouped together in a single embodiment, figure, or description thereof for the purpose of streamlining the disclosure and aiding in the understanding of or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment.

While some embodiments described herein include some, but not other features included in other embodiments, combinations of features of different embodiments are meant to be within the scope of the disclosure, and form different embodiments, as would be understood by the skilled person. For example, in the following claims, any of the claimed embodiments can be used in any combination.

Thus, while certain embodiments have been described, it will be appreciated that other and further modifications may be made thereto without departing from the spirit of the disclosure, and it is intended to cover all such modifications, enhancements, and other implementations, which fall within the true spirit and scope of this disclosure. To the maximum extent permitted by law, the scope of this disclosure is to be determined by the broadest permissible interpretation of the following claims, and shall not be restricted or limited by the foregoing detailed description

While various implementations of the disclosure have been described, it will be readily apparent to the skilled person that many more implementations are possible within the scope of the disclosure.

1. A method for determining the phase of gait of a subject, the method comprising:
transmitting from at least one motion sensor, data corresponding to motion of a part of a lower limb of the subject to a control unit;
estimating a gait phase of the subject using the control unit; and
sending an input signal based on the estimated gait phase to a controller;
based on the input signal, executing at least any one of a pre-determined instruction and an action.

2. The method for determining the phase of gait of a subject according to claim 1, wherein said at least any one of a pre-determined instruction and an action comprises applying functional electrical stimulation (FES) to the lower limb of the subject.

3. The method for determining the phase of gait of a subject according to claim 1, wherein the step of estimating the gait phase of the subject using the control unit comprises:
assigning a phase vector to data received by the control unit from the at least one motion sensor;
determining the probability of the phase vector lying within each of a number of pre-defined gait phases; and
estimating the gait phase of the subject by selecting the gait phase having the highest probability of the phase vector lying therein.

4. The method for determining the phase of gait of a subject according to claim 3, wherein the phase vector is assigned based on a combination of data captured by the said at least one motion sensor that is representative of linear and rotational motion in x, y and z axes.

5. The method for determining the phase of gait a subject according to claim 4, wherein the data received by the control unit from said at least one motion sensor is transformed through a neural network stored in non-volatile memory in communication with the control unit to transform the raw data from said at least one motion sensor into the phase vector.

6. The method for determining the phase of gait of a subject according to claim 5, wherein the neural network comprises a plurality of baseline gait phase vectors against which the phase vector is compared and assigned a similarity score, and wherein the similarity score is used to define a probability distribution over a plurality of gait phase labels to determine the likelihood of the phase vector lying within a gait phase represented by each of the gait phase labels.

7. The method for determining the phase of gait of a subject according to claim 6, wherein the method further comprises the step of selecting the gait phase label having the maximum probability of the phase vector lying therein.

8. The method for determining the phase of gait of a subject according to claim 7, wherein the selected gait phase identifies whether the gait phase of the subject is any one of stationary, in a stance phase, and in a swing phase.

9. The method for determining the phase of gait of a subject according to claim 8, wherein at least one of the stance phase and swing phase is sub-divided into a plurality of sub-gait phases represented by the gait phase labels.

10. The method for determining the phase of gait of a subject according to claim 9, wherein the stance phase of the subject's gait cycle is sub-divided into sub-phases 1-6.

11. The method for determining the phase of gait of a subject according to claim 10, wherein the swing phase of the subject's gait cycle is sub-divided into sub-phases 7-12.

12. The method for determining the phase of gait of a subject according to claim 10, wherein the step of applying FES to a lower limb of the subject comprises the FES controller applying FES at least one gait phase of the subject.

13. (canceled)

14. (canceled)

15. The method for applying FES to a subject according to claim 11, wherein the plurality of gait phase labels further comprises gait phase label 13 that is representative of the subject having any one of both feet on the floor and undertaking an activity that does not involve any one of walking and running.

16. A method of estimating gait phase of a subject, the method comprising:
transmitting from at least one motion sensor to a control unit, data corresponding to motion of a part of a lower limb of the subject to a control unit;
assigning a phase vector to the data received by the control unit from the at least one motion sensor;
determining the probability of the phase vector lying within each gait phase of the subject's gait cycle; and
estimating the gait phase of the subject by selecting the gait phase having the highest probability of the phase vector lying therein.

17. An apparatus for estimating phase of gait of a subject comprising:
at least one motion sensor associated with the limb of a subject;
a control unit;
non-volatile memory; and
communication circuitry,
wherein, the at least one motion sensor is configured to receive data associated with motion of the limb of the subject, said data being transmitted to the control unit and stored in the non-volatile memory, and wherein external data representative of an action to be undertaken at a predetermined phase of gait is receivable by the communication circuitry and stored in the non-volatile memory,
wherein the controller is configured to determine the phase of gait from the received data and undertake the action in accordance with the predetermined phase of gait.

18. The apparatus for determining phase of gait according to claim 17 further comprising a support substrate to which each of the at least one motion sensor, controller, non-volatile memory and communication circuitry are integrated, the support substrate being configured to selectively insert and remove an item of clothing thereto/therefrom.

19. The apparatus for determining phase of gait according to claim 18 further comprising a functional electrical stimulation (FES) apparatus for applying FES to the subject's limb.

20. The apparatus for determining phase of gait according to claim 19, wherein the FES apparatus is variable in terms of voltage and duration of FES application.

21. The apparatus for determining phase of gait according to claim 17, wherein the at least one motion sensor comprises at least one IMU.

22. The apparatus for determining phase of gait according to claim 18, wherein the at least one IMU is supported by a rigid backing plate.
